(19) United States
(12) Patent Application Publication
(10) Pub. No.: US 2002/0120405 A1
(43) Pub. Date: Aug. 29, 2002

(54) Protein data analysis

(76) Inventors: Aled Edwards, Toronto (CA); Cheryl Arrowsmith, North York (CA); Matthew Kimber, Toronto (CA); Mark Gerstein, North Haven, CT (US)

Correspondence Address: Patent Group, Foley, Hoag & Eliot LLP, One Post Office Square, Boston, MA, 02109-2170, US

(21) Appl. No.: 09/965,654

(22) Filed: Sep. 27, 2001

Related U.S. Application Data
(63) Continuation-in-part of application No. 09/671,817, filed on Sep. 27, 2000.

(51) Int. Cl. G06G 7/48; G06G 7/58; G06F 19/00; G01N 33/48; G01N 33/50

(52) U.S. Cl. 702/19; 703/11

(57) ABSTRACT

The disclosure includes a description of a method of data mining protein data. The method includes accessing data identifying respective outcomes associated with a set of proteins subjected to a set of conditions and analyzing the data based on the outcomes.

### REFERENCE TO RELATED APPLICATIONS

This application claims priority to co-pending U.S. application Ser. No. 09/671,817, filed on Sep. 27, 2000, entitled “Methods for Determining the Biochemical and Biophysical Properties of Proteins”. This application is incorporated by reference in its entirety herein.

### BACKGROUND

Genome sequencing projects are providing vast amounts of information. With the whole genome of many organisms, including humans, complete or nearing completion, the next challenge involves the characterization of these gene products, proteins. This flood of sequence information, coupled with recent advances, in molecular and structural biology have also lead to the concept of “structural proteomics” or “structural genomics”, the determination of three dimensional (3D) protein structures on a genome-wide scale. The 3D-structural information of proteins may be used to uncover clues to protein function difficult to detect from sequence analysis. This application of structural proteomics is, in part, driven by the realization that fewer than 30% of all predicted eukaryotic proteins have a known function.

While useful, analysis of the DNA sequence alone generally does not allow one to infer the structure or function of gene products unless the sequence has high homology to another gene of known function. Gene sequence information alone often does not provide a complete and accurate profile of protein function or structure. After transcription from DNA to RNA, the mRNA transcript may be spliced in different ways prior to translation into the protein. Following translation, many proteins are modified, for example, by the addition of one or more carbohydrate or phosphate groups. These modifications are important to the structure and function of the protein, but are not directly coded by that protein's gene. Thus, a single gene can code for many protein products. As a consequence, the proteome is far more complex than the genome.

The function of a protein derives from its 3D structure. Thus, a model of the 3D structure of a protein generally provides more information about function than does the sequence of the protein. For example, proteins with little sequence homology but high structural homology have often been found to have similar biochemical functions. The function of a protein often involves interaction with a small molecule, another protein or other biomolecule, such as a lipid, sugar, or nucleic acid. The interaction of the protein with its target molecule is determined by amino acid residues which are close in space due to the protein's 3D structure, allowing those residues to simultaneously interact with the target molecule. However, these amino acids may be distant according to the linear amino acid sequence.

One method of predicting a function for a new protein involves comparing the amino acid sequence of the predicted protein coding region, or open reading frame (ORF), against functionally assigned sequences in protein sequence databases. If significant sequence or motif homology is found between the ORF and a sequence of known function from the protein sequence database, it is assumed that the two sequences share the same, or similar, function. Unfortunately, most ORFs share little or no or only partial homology with a functionally assigned sequence. Thus, a large proportion of new ORFs are found to encode proteins of unknown function. In addition, for those ORFs that harbor some homology to another sequence, often the region of homology comprises only a small fraction of the total sequence, leaving the rest unknown.

The function of a new protein can often be predicted by determining its 3D structure using nuclear magnetic resonance (NMR) or X-ray crystallography. The structure, rather than the amino acid sequence, is then compared to known protein structures of assigned function. This information is collected in the Protein Data Bank (PDB), which can be searched to find homologous structural features of known proteins. If structural homologues are found, the new protein may be predicted to have a function similar to that of the homologue. In many cases confirmation of the predicted function can be readily determined experimentally. This method has the potential to be far more reliable than primary sequence comparisons, as proteins with little sequence homology may adopt similar 3D conformations that impart similar function. To date the PDB database contains relatively few unique protein structures (<2000) giving the database limited predictive powers.

A related use of structural proteomics information is to determine a sufficient number of 3D structures to define a “basic parts list” of protein folds. Most other structures could then be modeled from this basis-set using computational techniques. This analysis becomes feasible when a sufficient number of high-resolution, 3D protein structures have been determined to establish rules of how proteins fold into functional biological macromolecules.

As protein structure is a fundamental part of molecular biology and disease, structural proteomics will have an impact on many areas of biology including drug development. Application of structural proteomics to the pharmaceutical industry includes providing protein structural information for drug development, including identification and/or validation of new drug targets.

Historically, the explosion in gene sequence information has far outpaced the characterization of gene products. The processes of expressing and purifying proteins have represented a bottleneck in the efforts to obtain protein samples for 3-dimensional structure determination by NMR and X-ray crystallography. Generating high quality samples for structure determination by NMR or by X-ray crystallography (a well-behaving NMR sample or a well-diffracting crystal, respectively), is often perceived as a bottleneck in these efforts.

### SUMMARY

In general, in one aspect, the disclosure describes a method of data mining protein data. The method includes accessing data identifying respective outcomes associated with a set of proteins subjected to a set of conditions, and analyzing the data based on the outcomes.

Embodiments may include one or more of the following features. The outcomes may identify protein crystallization, protein solubility, or some intermediate form. The conditions may form a solution.

Analyzing may include determining the efficiency of a set of the conditions in producing a selected outcome in multiple ones of the proteins such as a subset of the proteins selected based on the similarity of characteristics of a protein with characteristics of proteins in the set of proteins.

The method may further include determining a prioritized set of conditions. Based on the set of conditions a kit of conditions may be provided.

The method may further include accessing data identifying characteristics of the protein. Analyzing the data may include analyzing the data based on the data identifying characteristics of the protein. The characteristics may include measured characteristics such as pI, secondary structure, amino-acid composition, oligometric state, protein mass, protein mono-dispersity. The characteristics may include determined characteristics such as protein sequence, amino acid composition, predicted pI, net charge, ratio of one or more pairs of amino acids, mass, predicted secondary structure, and predicted tertiary structure. The characteristics may include an encoding of the 3D structure of the protein, identification of the concentration of the protein, identification of a function of the protein, at least one location of the protein, and/or additives to the protein.

The method may further include accessing data identifying characteristics of different ones of the conditions. Analyzing the data may include analyzing the data based on the data identifying characteristics of the conditions such as pH.

### BRIEF DESCRIPTION OF THE DRAWINGS

FIG. 1 is a diagram of a decision tree for discriminating between soluble and insoluble proteins.

FIG. 2 is a diagram of data including a table identifying the outcome of different proteins under different conditions.

### DETAILED DESCRIPTION

Described herein are techniques that can use a database of protein data to derive a set of rules that are predictive of a given protein's biophysical and biochemical properties. The techniques described herein may also be used, for example, in a data mining process operating on protein/condition outcomes (e.g., protein crystallization, solubility, and other intermediate forms). Among a wide variety of other applications, such data mining may yield sets of conditions likely to yield a specified outcome for a protein. The proteins may include naturally occurring proteins, modified proteins, synthetic proteins and sub-domains of proteins.

A database may be constructed, for example, from protein sequence information and experimental data on protein biophysical and biochemical properties. The protein sequence information can include the primary amino acid sequence and characteristics which are derived from the sequence, including amino acid composition, the character of a region of the sequence, hydrophobicity, charge, molecular weight, the presence and length of low complexity regions and the presence of sequence motifs found in other proteins. The amino acid composition includes such information as the percent of a specific amino acid present in the sequence, the percent of a combination of two or more amino acids, and the percent of amino acids of a general class (such as, but not limited to, hydrophobic, hydrophilic, aromatic, aliphatic, acidic, basic, charged, and the like). Regions having a particular character may be, for example, regions of low sequence complexity, regions that are hydrophobic/hydrophilic, or charged regions (positive or negative). The source or the sequence information may be derived from the genomic DNA sequence, cDNA sequence, or synthetic DNA. The primary sequence information may come from a wide variety of sources, including human, animals, plants, yeast, bacteria, virus or engineered proteins.

The biophysical properties which populate the database may include, for example, thermal stability, solubility, isoelectric point, pH stability, crystallizability, conditions of crystallization, aggregation state, heat capacity, resistance to chemical denaturation, resistance to proteolytic degradation, amide hydrogen exchange data, behavior on chromatographic matrices, electrophoretic mobility and resistance to degradation during mass spectrometry. Biophysical properties may also include amenability (suitability) for study by various investigative techniques, including nuclear magnetic resonance (NMR), X-ray crystallography, circular dichroism (CD), light scattering, atomic adsorption, fluorescence, fluorescence quenching, mass spectroscopy, infrared spectroscopy (IR), electron microscopy, atomic force microscopy and any results obtained from these techniques. The conditions under which the property was determined may be incorporated into the database. These conditions may include solvent choice, protein concentration, buffer components and concentration, pH, temperature and salt concentration. It is advantageous to record a protein's properties determined under a variety of experimental conditions. Additional proteins are studied using the same set of conditions. In cases where applicable, negative information is recorded in the database (for example, insolubility, unsuitability for study by NMR, etc.) To insure uniformity of the data collected, it is preferred to perform the biophysical measurements on proteins that have been purified. It is especially preferable that the proteins are at least about 95% pure.

Among the biophysical properties which may be included in the database are those that relate to X-ray crystallographic techniques. These properties include conditions under which a protein does or does not crystallize, including solvents, precipitants, buffer components and concentration, pH, temperature, and salt concentration. The properties also include any results obtained from the X-ray crystallography studies, including three dimensional structure, characteristics of the crystal, including space group, solvent content, unit cell parameters, crystal contacts, solution conditions and bound water, and substrate binding. Additionally, the database may include how the various conditions employed effect results that are obtained.

The biochemical properties that comprise the database may include expressability, or level of expression in various vectors and hosts with various fusion tags and under various conditions, such as temperature and medium composition, the protein yield obtained from various vectors and hosts under various conditions, results of small molecule binding screens, subcellular localization, demonstrated utility as a drug target, and knowledge of protein-protein or protein-ligand interactions. A biochemical property of particular interest is the protein's potential as a drug target.

Some applications of these techniques may feature large numbers of proteins examined and compared under uniform conditions. The advent of high-throughput cloning and expression techniques and of high-throughput protein purification techniques has contributed to the feasibility of collecting this large volume of information. In theory, one might be able to compile the type of data listed above on a larger number of proteins from published accounts in the literature. Data from literature sources is not acquired under “standard” or uniform conditions. Furthermore, it is hard to assess the quality control or to fully ascertain the experimental conditions in many literature papers. Therefore, such a literature database would inherently yield less reliable predictions. For example, one can find data on protein yields from E. coli expression for many proteins. However, the conditions of growth (length of incubation time, temperature, induction condition, and so forth) are variable and can have effects on the experimental result. Thus, correlations between protein characteristics and expressability based on such data may lack reliability. Additionally, the intrinsic noise or scatter in the data might mask more subtle correlations.

For some applications, uniformity of the data may be preferable. For example, the biophysical and biochemical data are collected using a uniform set of conditions or experimental procedures. The conditions under which the empirical data are collected are recorded in the database. Ideally, multiple conditions are recorded for each type of measurement. The conditions of the data collection (temperature, solution components, salt concentration, buffer, pH) can drastically affect the behavior of a given protein. Therefore, it is desirable to compare many proteins under the same set of conditions, so that the only variable is the protein sequence. Alternatively, one can compare a variety of conditions for a give protein (or set of proteins) and relate that to sequence features.

In order to mine this data, it is annotated in the database using a “controlled vocabulary”. For example, data entry for solubility could be either a number, such as a quantitative measurement (for example, solubility in mg/ml), or a qualitative numerical scale (for example, a scale of 0-5, with 0 being completely insoluble, and 5 being very soluble). Direct instrumental measurements can also be used if internal calibration standards are used, so that the values can be related to some standard.

As a sufficient quantity of data is compiled in the database, the data can be analyzed using data-mining techniques, or knowledge discovery tools, for example, to find correlations among protein sequence information and biochemical or biophysical properties. These correlations can yield predictive rules for general protein behavior. The correlations may link protein sequence information alone, or in combination with one or more biochemical or biophysical properties, to a certain characteristic or a set of characteristics. Using the correlations obtained from the data-mining techniques, the properties of new proteins are determined given their amino acid sequence information alone or using a combination of the sequence information and one or more empirical properties.

Data-mining techniques, or knowledge discovery tools, include computer algorithms and associated software for identifying relationships between elements of the database. Data-mining techniques include, for example, decision-tree analysis, case-based reasoning, Bayesian classification, simple linear discriminant analysis, and support vector machines.

The predictive nature of the techniques described herein allows one to preemptively adjust experimental conditions to optimize, for example, cloning techniques, protein expression techniques, purification techniques and protein structure determination techniques. Thus, the invention provides a method for optimizing high-throughput protein structure determination. Using the predictive power of the empirical database in conjunction with data-mining tools, and the correlations obtained therefrom, the biochemical and biophysical properties of new proteins are predicted. Based upon these predictions, experimental conditions for the analysis of a protein, or class of proteins, is modified. Conversely, the invention provides a screening method to identify proteins that exhibit the desired properties for structural analysis or for use as a substrate for high-throughput drug screening. By the method of the invention, the biochemical or biophysical properties of new proteins are determined. Proteins that are determined to have a desired property or properties are then selected for further analysis. In this way, optimal proteins can be selected based on properties including one or more of crystallizability, suitability for NMR, expressability in a certain vector, solubility, suitability for study by a certain investigative technique and suitability for drug screens.

The techniques can speed up the high-throughput structure determination process. The 3D structure of a protein can also reveal whether it is likely to be a good drug target. Good drug targets generally, have deep, often hydrophobic, clefts or grooves on their surface or at their active sites where small molecule drugs can bind with high affinity. Poor drug targets have shallow grooves or otherwise poor surface properties that do not allow for high affinity binding of small molecules. By rapidly identifying which proteins have surface properties that make it promising for drug binding, the method greatly facilitates the drug discovery process.

The techniques can also provide a method to identify proteins that exhibit desired biochemical properties for drug interaction. Such biochemical properties may include the propensity to bind or interact with certain small molecules such as, for example, hydrophobic compounds, carbohydrates, or metal ions, or certain classes of drugs, pesticides, herbicide, or insecticides. Proteins that are determined to have a desired property or properties are then selected for further analysis. The screening of proteins as potential drug targets allows the researcher to selectively study proteins that are predicted to have desired biochemical or biophysical properties, thus reducing the research time and costs while greatly increasing the chance of success. The techniques may also provide a method of predicting which proteins are amenable to investigation as drug targets, thus speeding up the drug discovery process.

For example, the techniques can be used to predict from protein sequence information which proteins will be soluble and stable—a requirement for high-throughput biochemical screening of drug-target candidates. Thus, it greatly facilitates the development of high-throughput screening methods. Additionally, the techniques may be used to predict which proteins will crystallize and under what conditions, and which proteins will be amenable to NMR structure determination. The structure of a protein is useful in designing inhibitors or drugs that target that protein. The invention provides a rapid method of predicting which proteins are amenable to structure determination, thus speeding up the drug discovery process. In addition, the method of the invention will tell us which sequence features make a protein less amenable to structure determination, or less soluble and less stable. Thus, it provides the necessary knowledge to make point mutations, allowing the production of an analogous protein that will be more amenable to structure determination, or more soluble and more stable, again facilitating the target identification, validation and high-throughput screening and drug design processes. Certain classes of proteins, such as a specific enzyme class, may exhibit unique biochemical or biophysical properties. Thus, the invention can allow the creation of “class-specific” characteristics, which discover new members of the class or to modify members of the class to be more optimal in terms of activity, solubility, or suitability for structure determination.

Generally, the more protein characteristics compiled in the database, the greater the predictive powers achieved from the rules derived from the data-mining. For this reason the use of high throughput techniques in the assembly of the database is desirable. The wide availability of recombinant DNA technology makes it feasible to generate expression systems that can produce large quantities of a selected protein. The steps for protein production may include: generation of the protein expression systems, overexpressing the protein and purifying the protein.

The generation of a clone for any particular gene of interest, and its incorporation into a suitable expression vector, is now a straightforward task that can be done in a parallel fashion for high-throughput production. The selection of target proteins for structural analysis from completely sequenced genomes can take advantage of the availability of these cloned genes. However, even if a clone of a particular protein of interest is not readily available, it has now become a routine operation to generate a cDNA clone for almost any particular protein from a wide variety of organisms.

To obtain expression of a cloned nucleic acid, the expression vector for expression in bacteria contains a strong promoter to direct transcription, a transcription/translation terminator, and if the nucleic acid encodes a peptide or polypeptide, a ribosome binding site for translational initiation. Suitable bacterial promoters are well known in the art and described, e.g., in Sambrook et al. and Ausubel et al. Bacterial expression systems are available in, e.g., E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available. In certain cases, where post-translational modifications, for example, glycosylation are important, eukaryotic expression systems are preferred. In some cases, it may be preferable to employ expression vectors which can be propagated in both prokaryotic and eukaryotic cells, enabling, for example, nucleic acid purification and analysis using one organism and protein expression using another.

Transfection methods used to produce bacterial, mammalian, yeast or insect cells or cell lines that express large quantities of protein are well known in the art. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). After the expression vector is introduced into the cells, the transfected cells are cultured under conditions favoring expression of protein, which are then purified using standard techniques.

The protein may be expressed in suitable amounts for further analysis. There are several expression systems that have been extensively studied. Some of these include: 1) bacterial (E. coli), 2) methylotrophic yeast (Pichia pastorisis), 3) viral (baculovirus, adenovirus, vaccinia and some RNA viruses), 4) cell culture (mammalian and insect), and 5) in vitro translation. Although the expression of any particular protein may be idiosyncratic, the availability of these and other expression systems significantly increases the ability to produce large quantities of protein.

In situations in which relatively large amounts of relatively pure protein in native form are required, for example to obtain protein crystals useful for determination of 3D structure, it may be desirable to employ expression systems characterized by high expression levels, efficient protein processing including cleavage of signal peptides and other post-translational modifications. The baculovirus expression system is widely used to express a variety of proteins in large quantities. In addition to fulfilling the above requirements, the size of the expressed protein is not limited, and expressed proteins are typically correctly folded and in a biologically active state. Baclovirus expression vectors and expression systems are commercially available (Clontech, Palo Alto, Calif.; Invitrogen Corp., Carlsbad, Calif.).

Once a protein has been expressed to an acceptable level, the protein is purified from the other contents of the cell system that was utilized for expression. Highly purified protein is often desirable for further analysis according to the method of the invention. The proteins can be expressed fused to tags that aid subsequent purification or measurement techniques. Typical tags bind specifically to particular ligands, allowing the attached protein to be purified without regard to its physical or biochemical characteristics. Such tags can then be cleaved, leaving the protein in its native form. Examples of tags include histidine rich sequences that bind to various metal ions and glutathione-S-transferase (GST) tags which selectively bind to glutathione. The ligands are typically attached to a solid support. The fusion proteins are bound to the immobilized ligand and unbound material is removed. In certain cases, the fusion protein also includes a cleavable sequence of amino acids between the protein of interest and the tag sequence whereby the tag can be cleaved from the protein of interest. Typically, this is accomplished with a protease that cleaves the sequence under conditions where the protein of interest is not degraded, or with an intein sequence, which allows for internal cleavage of the protein. Alternatively, the tags can provide a method for specifically anchoring proteins to a solid support for assay purposes. For example, it can be useful to anchor proteins to an assay plate in order to measure fluorescence and fluorescence quenching in the presence of potential ligands. In another embodiment, a solid support is employed which provides an array of binding surfaces to which different proteins of the library are anchored for use in protein-ligand and protein-protein interaction studies. The solid support can be, for example, a glass or plastic plate, a semi-solid or gel-like matrix or the surface of a semiconductor measuring device. Bacterial vectors designed for production of GST fusion proteins are commercially available which allow cloning of DNAs in all three reading frames (e.g., pGEX series of vectors; Amersham Pharmacia Biotech, Inc., Piscataway, N.J.).

The following examples are provided as illustrative and are not limiting.

### EXAMPLE I

To explore the feasibility of a comprehensive structural proteomics project, 424 non-membrane proteins of unknown structure from Methanobacterium thermoautotrophicum are cloned, expressed in E. coli and purified. Using a single high-throughput protocol, about 20% of these are found to be suitable candidates for x-ray crystallographic or NMR spectroscopic analysis without further optimization of conditions, providing an estimate of the number of the most readily accessible structural targets in a proteome. A retrospective analysis of the empirical characteristics, including the experimental behavior, of these proteins provides some simple relations between sequence and biochemical and biophysical properties. A comprehensive database of protein properties is useful in optimizing high-throughput strategies.

Target Selection

M.th. is a thermophilic Archaeon whose genome comprises 1871 Open Reading Frames. Archaeal proteins share many sequence and functional features with eukaryotic proteins, but are often smaller and more robust, and thus serve as excellent model systems for complex processes. Only two exclusionary criteria were implemented in the target selection scheme. First, membrane-associated proteins, which comprise approximately 30% (267-422 of 1871 ORFs) of the M.th. proteome, were excluded. Second, proteins that have clear homologues in the PDB were excluded (approximately 27% of M.th. proteins). 424 of the remaining 900 final target M.th. proteins (almost a quarter of the entire proteome and a third of the non-membrane proteins) were chosen for cloning, expression and subsequent studies. These represent an unbiased sampling of non-membrane proteins from a single proteome with 34% having a functional annotation, 54% classified as “conserved” and 12% as “unknown”. This diverse collection of proteins was particularly valuable for retrospective analysis aimed at identifying sequence features that are predictive of protein biophysical and biochemical behavior.

Cloning Strategy

Each target gene was PCR-amplified from genomic DNA under standard, but optimized, conditions, with terminal incorporation of unique restriction sites, using high fidelity Pfu DNA polymerase (Stratagene). The PCR products were directionally cloned into the pET15b bacterial expression vector (NOVAGEN). The resulting plasmid encoded a fusion protein with an N-terminal hexa-histidine tag followed by a thrombin cleavage site. In the interest of throughput, no other expression vectors or organisms were used.

A single PCR protocol and set of cloning conditions were optimized for M.th. based on an analysis of an initial set of 50 genes. Positive clones were confirmed by colony PCR screening using Taq DNA polymerase. The generic nature of the procedure resulted in some PCR and sub-cloning failures, leading to a cumulative attrition rate of ˜6%. This protocol is readily scalable to 96-well format and has been extended to alternative vectors and expression organisms.

Expression Strategy

The M.th. open reading frames were divided arbitrarily into two groups, “large” (>20 kDa monomer size) and “small” (<20 kDa). Large proteins were processed for crystallization trials and small proteins for NMR feasibility studies. Most (˜80%) successfully cloned M.th. proteins could be expressed in E. coli BL21-Gold (DE3) cells (Stratagene), although efficient expression often required the presence of a second plasmid encoding three tRNAs which are frequently used by archeons and eukaryotes but are rare in E. coli. While most proteins were expressed to reasonable levels, many were not expressed in soluble form (<0.5 mg/L soluble protein), especially in the case of the larger proteins. It is possible to reduce the attrition rate due to poor solubility by optimizing the expression conditions for each clone. However, in the interest of throughput a single set of growth conditions optimized for the majority of proteins was used.

Purification and Crystallization of Large Proteins

For large proteins, three colonies from each transformation were tested for protein expression on a small scale (50 mL). Proteins found to be soluble by SDS-PAGE analysis of the bacterial extract were prepared on a larger scale (2 L). These proteins were purified by a combination of heat-treatment (55 C.) and nickel affinity chromatography, followed by thrombin cleavage and removal of the hexa-histidine tag. The heat treatment causes a significant enrichment of many, but not all, M.th. proteins. The purification of the proteins was monitored by denaturing gel electrophoresis and occasionally by mass spectrometry. Proteins that survived the purification process (˜75%) were concentrated to 10 mg/ml and subjected to a sparse-matrix crystallization screen of 48 conditions at room temperature (Matrix screen 1; Hampton Research). For each protein that crystallized in the initial screen, conditions were further optimized using an expansion of related solution conditions (typically 18-20 screens of 24 conditions for each protein). Twenty four of the proteins that formed crystals in the primary screen were followed up with optimization screens. Of these, 11 formed well diffracting crystals (<3.0 A). The implementation of automated methods for setting up and monitoring crystal screens can improve the throughput this process.

Purification and NMR Screening of Small Proteins

The smaller proteins (<20 kDa predicted monomer size) destined for NMR analysis were expressed five at a time, each in 1 L of ¹⁵N-enriched minimal media and purified in parallel using metal affinity chromatography. The resulting ¹⁵N-labeled hexahistidine fusion proteins were concentrated by ultrafiltration to ˜5-20 mg/ml, and the ¹⁵N-HSQC NMR spectrum taken at 25 C. The HSQC spectra were classified into one of three categories. The first, termed “excellent” and indicative of soluble, globular proteins, contained the predicted number of dispersed peaks of roughly equal intensity. These excellent spectra suggested that the process of determining their 3D structure is relatively straight-forward. The second type of spectrum, termed “promising”, had features such as too few or too many peaks and/or broad but dispersed signals. This suggested that optimization of either the protein construct or the solution conditions would be needed to yield an excellent sample. The last category, termed “poor”, comprised two kinds of spectra. The first, which have intense peaks but with little dispersion in the ¹⁵N-dimension, most likely reflect proteins that are soluble yet, largely unfolded. The second class had very low signal-to-noise and/or a single cluster of very broad peaks in the center of the spectrum. This class probably represented proteins that aggregate nonspecifically at concentrations required for NMR spectroscopy and thus were not readily amenable to structural analysis. For the 100 soluble proteins tested, the ratio of excellent/promising/poor spectra was 33/10/57.

Of the 33 proteins showing excellent spectra, seven were initially chosen for more detailed structure determination using NMR spectroscopy. For these samples the his-affinity tag was removed by proteolytic cleavage; this does not markedly change the spectral properties of the proteins, suggesting that this step may be omitted in the interest of saving time and maximizing protein yield. In one case (MTH40) it was necessary to further optimize solution conditions in order to prepare a sample that was stable for the time period (several weeks) necessary for NMR data collection.

### EXAMPLE II

### Analysis of Protein Folding and Stability by Circular Dichroism (CD) Spectroscopy

To explore how other spectroscopic techniques might aid in the identification of proteins suitable for detailed structural analysis, CD experiments were performed on 100 of the small, soluble MT proteins. Of the 28 proteins with excellent NMR spectra that a were re-examined, all but 6 displayed CD spectra that were typical of folded proteins containing a significant fraction of -helical and/or -sheet secondary structure. The six atypical spectra may have resulted from unusual structural features of the proteins in question (e.g. small-sheet proteins like SH3 domains possess very unusual CD spectra). Interestingly, 24 out of 32 proteins classified as “aggregated” by NMR spectroscopy displayed CD spectra consistent with stable, folded proteins. This suggested that the aggregation mechanism for many of the NMR samples was due to surface interactions in the folded state, as opposed to aggregation of the exposed hydrophobic cores of unfolded proteins. Knowledge of the aggregation mechanism is useful for optimizing solution conditions that disfavor aggregation and therefore, CD provides a useful secondary screen in structural proteomics projects.

To better understand the contribution of protein stability to sample behavior, the thermal unfolding of 60 folded MT proteins was analyzed. Of these, 22 were unfolded and refolded in a fully reversible manner. However, among the 19 proteins with “excellent” NMR spectra that were tested in this manner, only 9 refold reversibly. The others precipitated at high temperatures, demonstrating that even among well-folded, small, soluble proteins, reversible thermal unfolding in vitro was not a ubiquitous property. Surprisingly, 8 proteins classified as “aggregated” by NMR were well-behaved in thermal unfolding experiments, indicating that these proteins were probably large discrete oligomers rather than non-specific aggregates.

As expected for proteins from a thermophilic organism, those from M.th. all possessed high thermostability with transition midpoint temperature (Tₘ) values between 68 C. and 98 C. Due to their low change in heat capacity (Cₚ) upon unfolding, small proteins are generally expected to have higher Tₘ values compared to larger proteins. Here, however, no correlation between the length of the MT proteins and their T^{m} values was observed. The Cₚ values of small M.th. proteins were within the expected range as compared to a large number of other proteins that have been investigated. These data suggested that except for their high thermal stability, the overall thermodynamic behavior of M.th. proteins studied here may be representative of other mesophilic organisms.

### EXAMPLE III

### Retrospective Analysis of a Database of Biophysical and/or Biochemical Properties

The studies with Methanobacterium thermoautotrophicum revealed that poor expression and solubility accounted for almost 60% of the recalcitrant proteins. To identify the parameters that contributed to this poor sample behavior (and other factors related to suitability for expression, purification, and structure analysis), a retrospective data-mining approach was applied. Sequence data from the ˜424 proteins and the biophysical and biochemical data (expressability, crystallizability, solubility and melting temperature) were used to compile a database. Decision trees are useful for comprehensibly summarizing multivariate data and developing simple prediction rules. Growing the trees requires devising strategies regarding which variables (or combination of variables) to divide on, and what threshold to use to achieve the split. The 53 “splitting variables” used were derived from simple attributes of each sequence (e.g. amino acid composition, similarity to other proteins, measures of hydrophobicity, regions of low sequence complexity, etc.).

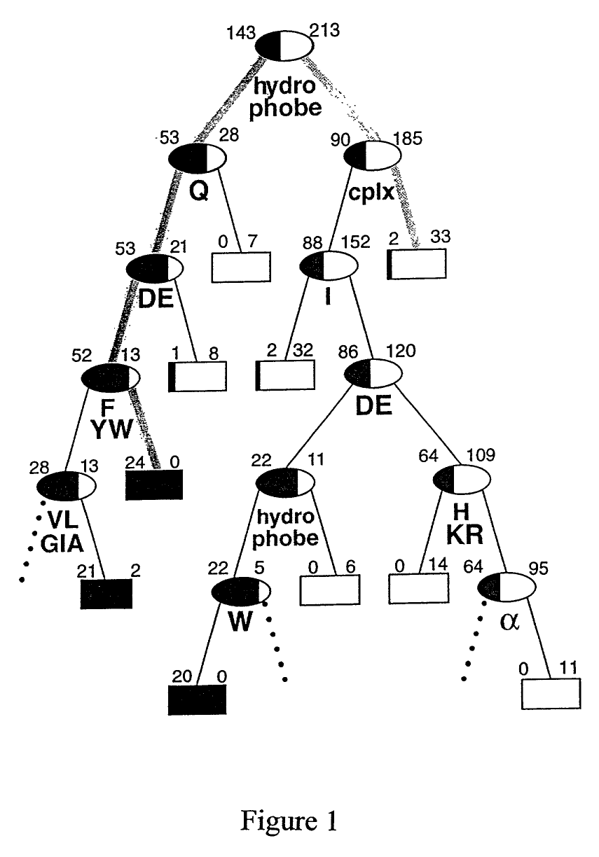

The full tree classifying the proteins according to their solubility (yes/no) had 35 final nodes and 65% overall accuracy in cross-validated tests. However, a number of the rules encoded within the tree were of much better predictive value. These are highlighted in FIG. 1.

FIG. 1 depicts a decision tree for discriminating between soluble and insoluble proteins. The nodes of the tree are represented by ellipses (intermediate nodes) and rectangles (final nodes or leaves). The numbers on the left of each node denote the number of insoluble proteins in the node, and are proportional to the node's dark area. Similarly, the numbers on the right denote the soluble proteins and are proportional to the white area. Under each intermediate node, the decision tree algorithm calculates all possible splitting thresholds for each of 53 variables (hydrophobicity, amino acid composition, etc.). It picks the optimal splitting variable and its threshold, in order for at least one of the two daughter nodes to be as homogeneous as possible. When a variable, v, is split, v<threshold is the left branch, and v>threshold is the right branch. The specific parameters used at each node and their thresholds for the right branches shown in the graph are in descending order (from top root to bottom leaves): hydrophobe>0.85 kcal/mole (where “hydrophobe” represents the average GES hydrophobicity of a sequence stretch, the higher this value the lower is the energy transfer); cplx>0.28 (a measure of a short complexity region based on the SEG program); Gln composition>4%; Asp+Glu composition>17%; Ile-composition>5.6%; Phe+Tyr+Trp composition>7.5%; Asp+Glu composition>13.6%; Gly+Ala+Val+Leu+Ile composition>42%; hydrophobe>0.01 kcal/mole; His+Lys+Arg composition>12%; Trp composition>1.2%; and alphahelical secondary structure composition>58%. Note that two of the variables are conditioned on more than once (hydrophobe, Asp+Glu). The highlighted decision pathways terminate in highly homogeneous nodes (mostly dark is insoluble, mostly white is soluble). The shorter the decision pathway and the larger the number of cases in the terminal node, the less likely it is to over-fit the data. Heterogeneous leaves could be further split (dotted lines) improving the error rate but risking over-fitting of the training set. The usual technique for assessing the predictive success of rules suggested by the tree in the context of overfitting is cross-validation, where the overall data set is divided into test and training components. However, this technique is not optimal on the relatively small samples associated with each rule in these trees, as one has to leave out a substantial fraction of information in devising each rule. The predictive values of the highlighted decision pathways are evaluated using a “pessimistic estimation” procedure which assumes that the error rate at each node is bionomially distributed, and then inflates the rate found on a tree based on all the data (by −2 standard deviations) to arrive at a more realistic estimate.

Proteins that fulfill the following sequence of four conditions are likely to be insoluble: (1) have a hydrophobic stretch—a long region (>20 residues) with average hydrophobicity less than −0.85 kcal/mole (on the GES scale); (2) Gln composition<4%; (3) Asp+Glu composition <17%; and (4) aromatic composition>7.5%. This rule has a 14% error rate in comparison to the default error rate of 39% for choosing a soluble protein without the aid of the tree. The probability that it could arise by chance is 1%, assuming one randomly chose the 24 insoluble proteins from the initial pool of 143 insoluble and 213 soluble proteins. These calculations are based on a “pessimistic estimate for errors”, taking the upper bound of the 95% confidence interval. Conversely, proteins that do not have a hydrophobic stretch and have more than 27% of their residues in (hydrophilic) “low-complexity” regions are very likely to be soluble. This rule has a “pessimistic” error rate of 20% in contrast to 39% without the tree and a 1% probability of occurring by chance.

Similar trees for expressability and crystallizability were derived. The composition of Asn appeared to be relevant to crystallizability. In particular, an Asn threshold of 3.5% was able to select a set of 18 crystallizable and only one non-crystallizable protein from the initial set of 25 crystallizable and 39 non-crystallizable proteins.

The techniques described herein have a wide variety of applications. For example, as described above, proteins have a wide variety of uses in their different forms. For instance, experiments often use proteins in soluble form. Other protein uses depend on crystallized proteins. For example, many proteins, such as insulin, are best delivered in crystallized form. As described above, crystallized proteins are also used in structural proteomics. Finding a useful protein crystal, however, can be a time and resource-consuming task. One strategy in obtaining a crystal involves screening a wide variety of solution conditions in the hopes of identifying conditions that will support crystallization. Unfortunately, the conditions that may cause one protein to crystallize, leave another protein soluble. The time and cost of determining suitable conditions that yield a desired outcome may pose a significant obstacle when multiplied over the hundreds or even thousands of proteins of interest.

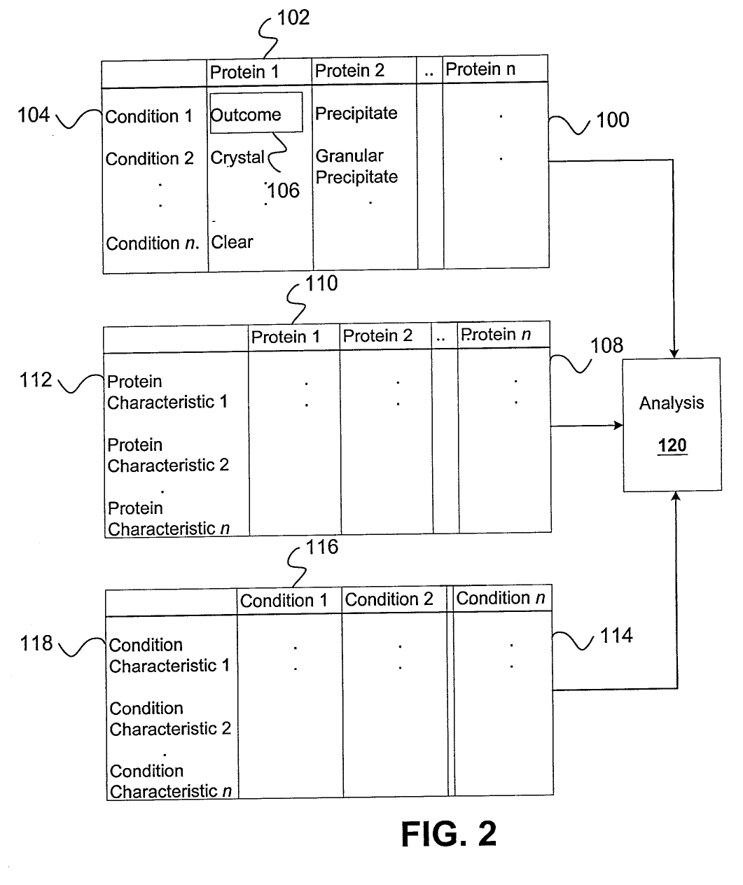

FIG. 2 illustrates an example of a data system that operates on the outcome (e.g., outcome 106) of a given protein 102 when subjected to a given condition 104. The outcome 106 can be categorized, for example, as crystal, as soluble, or in some intermediate form such as precipitate or granular precipitate. Analysis 120 of this data 100, and potentially other data such as characteristics of the proteins 108 and/or of the conditions 114, can yield a wide variety of useful information. For example, analysis 120 can predict an outcome of a new protein of interest subjected to a particular condition 114 based on the similarity of characteristics of the new protein with characteristics of other proteins. The data 100, 108, 114 can also identify relationships between characteristics of proteins and/or conditions that tend to lead to a particular outcome. By acting on predictive rules derived from the analysis 120, researchers can enjoy a greater likelihood of success of obtaining a desired protein form with less guesswork. This may be particularly important when working with a scarce protein.

In greater detail, FIG. 2 shows a system that includes a database table 100 that indicates the outcomes of different proteins 102 in different conditions 104. For example, the conditions 104 may include conditions 104 of the Jancarik and Kim screen (Jankarik, J. & S. H. Kim. J. Appl. Cryst., 1991. 24: p. 409-411). The outcomes may be determined based on human visual classification. The outcomes may also be determined via a machine system. Such a system may make finer gradations in the determining of outcome or include information about the number, size, and/or morphology of crystals. The machine may also operate at different wavelengths—such as U.V., where proteins absorb strongly, or x-rays, where proteins diffract.

The system may also include a table 108 that lists different characteristics 112 of different proteins 110. Since characteristics 112 of a protein may contribute heavily to outcomes under different conditions, a system may use this information to probabilistically correlate one or more protein characteristics 112 with crystallization or some other specified outcome.

The protein characteristics 112 may include empirically measured characteristics such as pI, secondary structure, amino-acid composition, oligometric state, protein mass, and/or protein mono-dispersity. The characteristics may also include determined characteristics such as characteristics derived from the protein sequence. These determined characteristics may include protein sequence, amino acid composition, predicted pI, net charge, ratio of one or more pairs of amino acids, mass, predicted secondary structure, and/or predicted tertiary structure. Such characteristics 112 may also include an encoding of the 3D structure of the protein (e.g., a mathematical encoding of the protein's surface), identification of the concentration of the protein, identification of a function of the protein, and/or identification at least one location of the source of the protein (e.g., organ, tissue or sub-cellular localization). The characteristics 112 may also include identification of additives (e.g., salts, buffers, and organic molecules).

The protein-condition outcome 106 may also depend on aspects of the condition. Thus, the system may further include data 114 that identifies characteristics 118 of conditions 116 used in table 100. For example, the table 114 may include characteristics 118 representing the contents of the condition 114 and/or the properties (e.g., pH) of a condition 114. The use of condition data may be used, for example, to identify conditions 114 highly correlated with a specified outcome. Additionally, such data may be used to improve a given set of conditions. For example, some of the conditions of the widely used Jancarik and Kim screen may be highly correlated in that if a protein crystallizes in one of the conditions, then it is also highly likely to crystallize in the other. Eliminating such redundancy can increase the overall efficiency of the screen and allows a wider diversity of conditions to be experimented with using the same amount of protein material. Thus, such data may lead to a screen that crystallizes more proteins while consuming a similar amount of material.

Analysis 120 of the data 100, 108, 114 may proceed in many different ways. For example, the data may be analyzed to determine the efficiency of conditions in producing a selected outcome for some subset of proteins. For instance, the condition 104 outcomes for proteins sharing a set of characteristics 112 may be aggregated to determine a likelihood of attaining a particular outcome, for example, for a new protein of interest having these characteristics. This can reveal conditions more suited to producing a specified outcome. These conditions may be prioritized to identify those conditions with the greatest efficiency in yielding the desired outcome. This can result in the conservation of the amount of protein needed to obtain a desired form. A kit of conditions may be pre-fabricated for use by researchers based on these results. For example, after determining a prioritized set of conditions that maximize efficiency of crystallization, a kit including the top n conditions may be assembled for distribution. After a similar process, a kit including the top n conditions for maximizing the efficiency of solubility may be assembled, and so forth.

Similar to the process described above in conjunction with protein characteristics, data analysis may operate on the condition characteristics, for example, to identify condition characteristics likely to yield a particular outcome. The process may also operate on combinations of protein and condition characteristics, for example, to identify combinations of protein characteristics and condition characteristics likely to yield a specified outcome.

The data 100, 108, 114 may be analyzed in a wide variety of ways and used for a variety of purposes. For example, patterns of solubility may act as a “diagnostic” of the protein's behavior in ADME-tox, assays, and protein interaction studies. Similarly, patterns that result in solubility outcomes may be used to derive functional information about the protein such as small molecule bindings.

More specifically the data 100, 108, 114 may be analyzed to determine one or more of the following: a prioritized set of conditions to maximize efficiency of crystallization of a protein; a prioritized set of conditions to maximize protein solubility of a protein; information (e.g., a predictive rule) which relates aspects of a protein that may be derived from knowledge of the sequence to protein solubility; information which relates aspects of a protein that may be derived from knowledge of the protein sequence to protein crystallization; information that relates at least one experimentally measurable property of a protein sample to protein crystallization; information that relates some experimentally measurable property of a protein sample to protein solubility; information that relates pH to protein solubility; information that relates the concentration and chemical nature of additives to protein solubility; information that relates a protein's 3D structure to protein solubility; information that relates protein concentration to protein crystallization; information that relates a protein's function to protein solubility; and/or information that relates a protein's solubility behavior to that protein's organ, tissue or sub-cellular localization.

The analysis 120 may feature a variety of data mining tools such as statistical techniques that determine the interdependence of variables on protein-condition outcome. For example, statistical regressions may be run to identify protein and condition characteristics or sets of characteristics that highly correlate with crystallization, solubility, or other specified form. Additionally, the data mining techniques described above, among others, may also be integrated.

The techniques described herein are not limited to any particular hardware or software configuration; they may find applicability in any computing or processing environment. The techniques may be implemented in hardware or software, or a combination of the two. Preferably, the techniques are implemented in computer programs executing on programmable computers that each include a processor, a storage medium readable by the processor (including volatile and non-volatile memory and/or storage elements), at least one input device, and one or more output devices.

Each program is preferably implemented in high level procedural or object oriented programming language to communicate with a computer system. However, the programs can be implemented in assembly or machine language, if desired. In any case the language may be compiled or interpreted language.

Each such computer program is preferably stored on a storage medium or device (e.g., CD-ROM, hard disk, or magnetic disk) that is readable by a general or special purpose programmable computer for configuring and operating the computer when the storage medium or device is read by the computer to perform the procedures described herein. The system may also be considered to be implemented as a computer-readable storage medium, configured with a computer program, where the storage medium so configured causes a computer to operate in a specific and predefined manner.

Other embodiments are within the scope of the following claims.

What is claimed is:

1. A method of data mining protein data, the method comprising:
accessing data identifying respective outcomes associated with a set of proteins subjected to a set of conditions; and
analyzing the data based on the outcomes.

2. The method of claim 1, wherein one of the outcomes comprises identification of protein crystallization of one of the set of proteins in one of the set of conditions.

3. The method of claim 1, wherein one of the outcomes comprises identification of protein solubility of one of the set of proteins in one of the set of conditions.

4. The method of claim 1, wherein at least one of the conditions comprises a solution.

5. The method of claim 1, wherein analyzing comprises determining the efficiency of a set of the conditions in producing a selected outcome in multiple ones of the proteins.

6. The method of claim 5, wherein the multiple ones of the proteins comprises a subset of the set of proteins.

7. The method of claim 6, further comprising selecting the subset of the set of proteins.

8. The method of claim 7, wherein selecting comprises selecting based on the similarity of characteristics of a protein with characteristics of proteins in the set of proteins.

9. The method of claim 5, further comprising determining a prioritized set of conditions.

10. The method of claim 5, further comprising providing a kit on conditions based on the determining.

11. The method of claim 1,
further comprising accessing data identifying characteristics of the protein; and
wherein analyzing the data comprising analyzing the data based on the data identifying characteristics of the protein.

12. The method of claim 11, wherein the characteristics comprise measured characteristics.

13. The method of claim 12, wherein the measured characteristics comprise at least one of the following: pI, secondary structure, amino-acid composition, oligometric state, protein mass, and protein mono-dispersity.

14. The method of claim 11, wherein the characteristics comprise determined characteristics.

15. The method of claim 14, wherein the determined characteristics comprise at least one of the following: protein sequence, amino acid composition, predicted pI, net charge, ratio of one or more pairs of amino acids, mass, predicted secondary structure, and predicted tertiary structure.

16. The method of claim 11, wherein the characteristics comprise an encoding of the 3D structure of the protein.

17. The method of claim 11, wherein the characteristics comprise identification of the concentration of the protein.

18. The method of claim 11, wherein the characteristics comprise identification of a function of the protein.

19. The method of claim 11, wherein the characteristics comprise at least one location of the protein.

20. The method of claim 11, wherein the characteristics comprise additives to the protein.

21. The method of claim 11,
further comprising accessing data identifying characteristics of different ones of the conditions; and
wherein analyzing the data comprising analyzing the data based on the data identifying characteristics of the conditions.

22. The method of claim 21, wherein the condition characteristics comprise pH.

23. A computer program product, disposed on a computer readable medium, for data mining protein data, the computer program product including instructions for causing a processor to:
access data identifying respective outcomes associated with a set of proteins subjected to a set of conditions; and
analyze the data based on the outcomes.

24. The computer program of claim 23, wherein one of the outcomes comprises identification of protein crystallization of one of the set of proteins in one of the set of conditions.

25. The computer program of claim 23, wherein one of the outcomes comprises identification of protein solubility of one of the set of proteins in one of the set of conditions.

26. The computer program of claim 23, wherein the instructions for causing the processor to analyze comprise instructions for causing the processor to determine the efficiency of a set of the conditions in producing a selected outcome in multiple ones of the proteins.

27. The computer program of claim 26, wherein the multiple ones of the proteins comprises a subset of the set of proteins.

28. The computer program of claim 27, further comprising instructions for causing the processor to select the subset of the set of proteins.

29. The computer program of claim 28, wherein the instructions for causing the processor to select comprise instructions for causing the processor to select based on the similarity of characteristics of a protein with characteristics of proteins in the set of proteins.

30. The computer program of claim 23,
further comprising instructions for causing the processor to access data identifying characteristics of the protein; and
wherein the instructions for causing the processor to analyze the data comprise instructions for causing the processor to analyze the data based on the data identifying characteristics of the protein.

31. The computer program of claim 23,
further comprising instructions for causing the processor to access data identifying characteristics of different ones of the conditions; and
wherein the instructions for causing the processor to analyze the data comprise instructions for causing the processor to analyze the data based on the data identifying characteristics of the conditions.
